(19) United States
(12) Patent Application Publication
(10) Pub. No.: US 2017/0135637 A1
(43) Pub. Date: May 18, 2017

(54) REAL-TIME GLUCOSE MONITORING

(71) Applicants: Zhen XIAO, Beijing (CN); EMPIRE TECHNOLOGY DEVELOPMENT LLC, WILMINGTON, DE (US)

(72) Inventor: Zhen XIAO, Beijing (CN)

(73) Assignee: EMPIRE TECHNOLOGY DEVELOPMENT LLC, WILMINGTON, DE (US)

(21) Appl. No.: 15/322,227

(22) PCT Filed: Jul. 31, 2014
(86) PCT No.: PCT/CN2014/083391

§ 371 Date: Dec. 27, 2016

(51) Int. Cl. A61B 5/00; A61B 5/1455; A61B 5/145

(52) U.S. Cl. CPC A61B 5/6821; A61B 5/14532; A61B 5/1455; A61B 5/14507

(57) ABSTRACT

Embodiments pertaining to techniques of real-time glucose monitoring in tears are provided. In some examples, a device may include a plug and a sensor. The plug may be adapted for placement in a lacrimal punctum of an eyelid of a subject. The sensor may be associated with a head of the plug and may be in contact with the tears. The sensor may be adapted for measurement of a concentration of an analyte in the tears.

### TECHNICAL FIELD

The embodiments described herein pertain generally to monitoring of an analyte in tears and, more particularly, to real-time glucose monitoring in tears.

### BACKGROUND

Unless otherwise indicated herein, the approaches described in this section are not prior art to the claims in this application and are not admitted to be prior art by inclusion in this section.

Tear fluid is the aqueous layer on an ocular surface. Tear fluid has various functions, such as controlling infectious agents, lubricating the eye, and nourishing the cornea. Tear fluid normally includes various chemical compounds, such as salt water, proteins, glucose, some small metallic ions, etc. Among these compounds, tear glucose has been studied for diabetes diagnostics because, similar to blood glucose levels, tear glucose levels are higher in diabetic subjects than in healthy ones. In addition, the correlation between tear glucose and blood glucose has been studied and demonstrated in both human and animals. However, sufficient amount of tears suitable for a conventional glucose assay takes long time to collect, and tear glucose levels are lower than those in blood. This causes some of the conventional assays for measuring levels of blood glucose not to be suitable for tear glucose.

### SUMMARY

In one example embodiment, a device may include a plug adapted for placement in a lacrimal punctum of an eyelid of a subject, and a sensor that is associated with a head of the plug and is in contact with the tears. The sensor may be adapted for measurement of a concentration of an analyte in the tears.

In another embodiment, a method may include: providing a plug including a sensor adapted for measurement of a concentration of an analyte in tears, and placing the plug in a lacrimal punctum of an eyelid of a subject. The sensor may include a fluorescence resonance energy transfer (FRET) system indicative of presence of the analyte in the tears.

In yet another example embodiment, a system may include a plug adapted for placement in a lacrimal punctum of an eyelid of a subject having diabetes, and a receiver adapted for receiving a fluorescence emission signal. The plug may include a sensor that is associated with a head of the plug and in contact with tears. The sensor may be adapted for measurement of a concentration of glucose in the tears and may include a FRET system. The FRET system may include a pair of fluorophores. The system may also include a light source adapted to excite the FRET system to generate the fluorescence emission signal when a fluorophore of the pair of fluorophore is excited by a light source, and an analyte binding moiety associated with another fluorophore of the pair of fluorophores binds the analyte.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

In the detailed description that follows, embodiments are described as illustrations only since various changes and modifications will become apparent to those skilled in the art from the following detailed description. The use of the same reference numbers in different figures indicates similar or identical items.

FIG. 1 shows a framework which enables real-time glucose monitoring in tears using a fluorescence resonance energy transfer (FRET) system, arranged in accordance with at least some embodiments described herein;

FIG. 2 shows an example scheme illustrating mechanism of a FRET system, arranged in accordance with at least some embodiments described herein;

FIG. 3 shows an example processing flow with which a concentration of an analyte in tears of a subject may be calculated and monitored, arranged in accordance with at least some embodiments described herein.

### DETAILED DESCRIPTION

In the following detailed description, reference is made to the accompanying drawings, which form a part of the description. In the drawings, similar symbols typically identify similar components, unless context dictates otherwise. Furthermore, unless otherwise noted, the description of each successive drawing may reference features from one or more of the previous drawings to provide clearer context and a more substantive explanation of the current example embodiment. Still, the example embodiments described in the detailed description, drawings, and claims are not meant to be limiting. Other embodiments may be used, and other changes may be made, without departing from the spirit or scope of the subject matter presented herein. It will be readily understood that the aspects of the present disclosure, as generally described herein and illustrated in the drawings, may be arranged, substituted, combined, separated, and designed in a wide variety of different configurations, all of which are explicitly contemplated herein.

There are conventional methods for measuring glucose of diabetes patients in a non-invasive manner. One of such methods involves measuring the glucose concentration in tears from which the concentration of blood glucose may be estimated. This method includes installing a measuring device on a contact lens to determine a tear glucose level. However, this method is not appropriate for diabetes patients since the diabetes patients are typically not suitable to wear contact lenses due to their poor resistance and diminished functions of sensory nerves on a corneal surface. In fact, wearing contact lenses may increase the possibility of corneal infection in a diabetes patient. In addition, a contact lens employing a circuit to measure tear glucose levels is even less appropriate for daily wear and use by diabetes patients. The circuit occupies some oxygen permeation areas of the lens, and therefore the oxygen permeation capability of the lens is further reduced.

Embodiments of the present disclosure use a plug (for example, a modified punctal plug) as a device for measuring tear glucose levels for diabetes patients. The head of the modified punctal plug exhibits fluorescence with different intensities in response to different glucose concentrations, and thus a tear glucose concentration may be determined based on the fluorescence. In some embodiments, the plug may be placed or implanted in an eyelid (for example, a lacrimal punctum), resulting in sustainable non-invasive measurement of glucose levels in tears while not affecting oxygen supply to ocular tissues. In addition, the opening of the lacrimal punctum is usually in contact with the eye wall instead of being directly exposed to sunlight; so fluorescence material of the plug is substantially protected from fluorescence quenching.

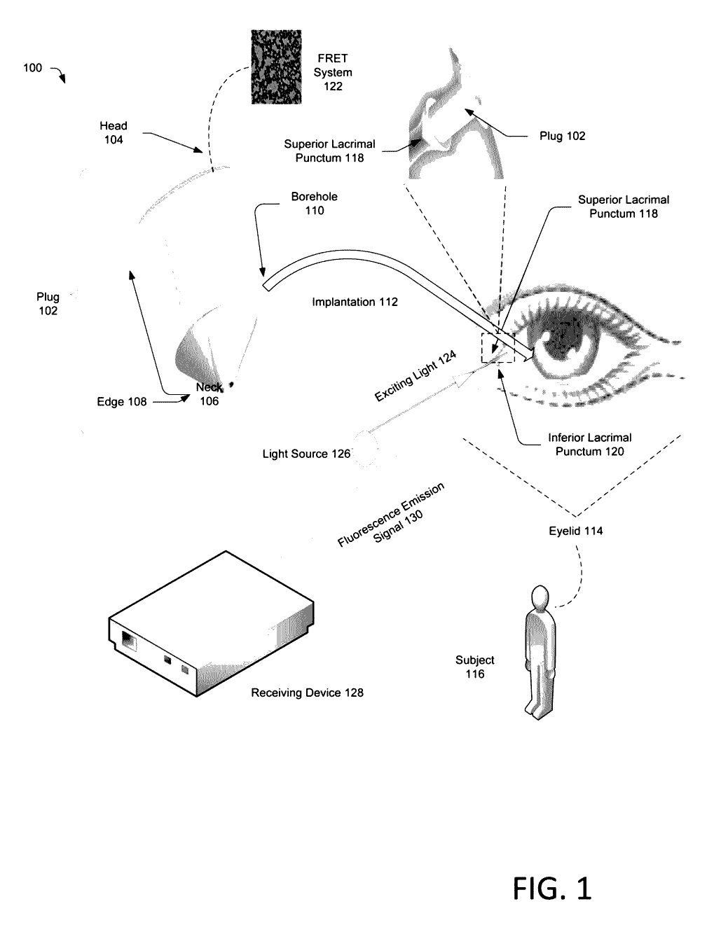

FIG. 1 shows a framework 100 which allows real-time glucose monitoring in tears using a fluorescence resonance energy transfer (FRET) system, arranged in accordance with at least some embodiments described herein. Framework 100 includes a plug 102, which includes various components, such as a head 104, a neck 106, an edge 108, and a borehole 110. Plug 102 may be adapted for placement in a lacrimal punctum of an eyelid of a subject. For example, plug 102 may be placed (for example, via implantation 112) into an eyelid 114 of a subject 116. In some embodiments, plug 102 may be implanted into a superior lacrimal punctum 118 and/or an inferior lacrimal punctum 120. For example, as illustrated in FIG. 1, plug 102 is implanted into superior lacrimal punctum 118.

In some embodiments, at least a portion of plug 102 (for example, head 104) may include at least one of the following: silicone acrylates, silicone derivatives, fluorophore, polymethylmethacrylate (PMMA), polydimethylsiloxane (PDMS), or polydimethylsiloxane. In some embodiments, plug 102 may be made from silicone or silica aerogel and may have a morphology similar to punctal plugs that are made using conventional techniques.

In some embodiments, the plug may be a punctal plug adapted for placement in a lacrimal punctum. In some instances, plug 102 may be a rod-like structure with a diameter of about 0.5 mm to about 0.8 mm and may be stuffed into a lacrimal punctum at a nasal side of eyelid of a patient (for example, subject 116). Plug 102 may be used to obstruct the entrance of the lacrimal canaliculi to keep tears staying longer in eyes. The inferior lacrimal punctum of an eye may receive about 75% of the tear volume, and the superior lacrimal punctum may receive about 25% of the tear volume. In some embodiments, plug 102 may be used to relieve mild to moderate xerophthalmia of subject 116. In these instances, if subject 116 has diabetes and xerophthalmia, plug 102 may be implanted into inferior lacrimal punctum 120. If subject 116 has diabetes but no xerophthalmia, then plug 102 may be implanted into superior lacrimal punctum 118 to cause little effect on discharge of tears of subject 116.

Head 104 may embed a sensor adapted for measurement of a concentration of an analyte (for example, glucose) in tears of subject 116. In some embodiments, the sensor may include FRET system 122 indicative of presence of the analyte in the tears in eyelid 114. In some instances, subject 116 may have diabetes, and the sensor may detect and measure a glucose level (for example, a concentration of the glucose) in the tears in eyelid 114. For example, FRET system 122 may be adapted for a fluorescence-based chromatographic assay, and a glucose level in the tears of subject 116 may be determined based on the fluorescence-based chromatographic assay.

In some embodiments, FRET system 122 may be encapsulated into a membrane containing physiologically compatible porous nanostructures such that FRET system 122 may be substantially retained on or within the physiologically compatible porous nanostructures. In these instances, the physiologically compatible porous nanostructures may include fluorescent mesoporous silica nanoparticles (FMSN). In some embodiments, the physiologically compatible porous nanostructures may include a porous medium adapted for collection of the tears.

For example, FRET system 122 may be encapsulated within the FMSN using various methods, such as water-in-oil microemulsion method, sol-gel method, etc. In some embodiments, the average diameter of the nanoparticles is around 55±10 nm. The overall reaction of producing FMSNs is expressed by Equation 1 below.

Si(OR)₄+2H₂O→SiO₂+4EtOH (1)

FMSN may be firmly attached to a punctal plug material of plug 102. For example, pre-prepared nanoparticles may be added to a silica sol. The mixed sol may then be gelatinized into a mixed wet gel after a certain time period (for example, several minutes or dozens of seconds). Then, other processes including desolvation and annealing may be performed to obtain a nanocomposite. Since the silica sol is gelatinized in a short time period after mixing with the nanoparticles, the silica sol may form a reticular structure. In the reticular structure, the nanoparticles may be distributed and be restricted from growing up. Accordingly, the resulting composite may maintain characteristics of mesoporous silica, which has a large surface area and high porosity. Also, the resulting composite may contain processed nanoparticles, which maintain size and morphology similar to those of the pre-prepared nanoparticles. In these instances, the processed nanoparticles are not coated by silica and therefore may communicate directly with environment outside of plug 102. The resulting composite (for example, nanocomposite s) can be recut or poured into a mold during gelation to form at least a portion of plug 102.

In some embodiments, FRET system 122 may include a pair of fluorophores such that FRET system 122 generates a fluorescence emission signal when a first fluorophore of the pair of fluorophores is excited by a light source, and an analyte binding moiety of FRET system 122 binds the analyte. In these instances, the analyte binding moiety may be contained in FRET system 122 and associated with an additional fluorophore of the pair of fluorophores. In some instances, the first and second fluorophores of the pair of fluorophores differ by at least about 30 nm in terms of fluorescent wavelengths. In some embodiments, the fluorophores of the pair of fluorophores may include at least one of the following: rhodamine and fluorescein isothiocyanate (FITC), tetramethyl rhodamine isothiocyanate (TRITC) and FITC, or tetramethylrhodamine (TAMRA) and FITC.

Plug 102 may be placed in a lacrimal punctum of eyelid 114 of subject 116 to monitor an analyte level of the tears of subject 116. In some embodiments, FRET system 122 may be excited by exciting light 124, which is generated by at least one light source 126. In these instances, at least one receiving device 128 may detect and/or receive a fluorescence emission signal 130 generated by FRET system 112. For example, fluorescence emission signal 130 may include information associated with a fluorescence intensity, a fluorescence wavelength, and/or a fluorescence lifetime.

Based on the fluorescence emission signal 130, a concentration of the analyte in the tears may be calculated. For example, receiving device 128 may include or be associated with at least one detector, which may be in optical communication with FRET system 122. The detector may be configured and arranged to detect at least a wavelength of emission light from the sensor embedded in head 104. The detector may include at least one of the following: a photodiode having an interference filter, a prism or grating having a charge-coupled device (CCD) array detection element, a photomultiplier tube. For example, the detector may be made and/or integrated into various devices, such as a hand-held device (for example, ChromalD® technology from Visualant®), a device associated with a mirror, a device integrated into a spectacle frame, etc.

For example, as a hand-held device, the device may include an annular eye pad, which contacts with an orbit of subject 116′. When subject 116 presses the annular eye pad closely to the orbit, a projection near the nasal side of the eye pad may gently compress the skin near the nasal side of eyelid 114, resulting in ectropion and exposure of superior lacrimal punctum 118 and/or inferior lacrimal punctum 120. Light source 126 may be associated with the hand-held device and emit excitation light 124 to irradiate the lacrimal punctum position and to excite nanoparticles (for example, FRET system 122) on the surface of head 104. Then, the generated fluorescence (for example, fluorescence emission signal 130) may be collected and/or measured by, for example, a CCD.

In some embodiments, the detector may be installed on a mirror. For example, the detector may be hidden behind a make-up mirror. Subject 116 may compress the nasal side of eyelid 114 gently with fingers to make head 104 exposed and may then get close to the mirror. Subject 116 may then gaze on a cross marker line on the mirror surface, align the mirror image of the eye, and gradually get close to the mirror. In some instances, an ultrasonic distance sensor may be used to ensure that the position of the eyes of subject 116 remains substantially unchanged, and that a measurable position is reached. Light source 126 may be associated with the detector and emit excitation light 124 to irradiate the lacrimal punctum position and to excite nanoparticles (for example, FRET system 122) on the surface of head 104. Then, the generated fluorescence (for example, fluorescence emission signal 130) may be measured by, for example, CCD.

In some embodiments, the detector may be integrated into a spectacle frame. For example, subject 116 may press the nose pads of the spectacle frame gently to make the inferior lacrimal punctum exposed and trigger fluorescence measurement, which is similar to those discussed above. In some examples, one or more excitation light sources supported by the frame irradiate one or both eyes around a sensor position, and then the fluorescence generated is measured by one or more photosensors, such as a photodiode, CCD, and the like which may also be supported (for example part of) the frame. For example, one or both lens frames of the spectacles may include one or more light emitting diodes (LEDs) of different wavelengths, which may be sequentially energized and corresponding signals from the photodetector analyzed to characterize the sensor. The frame may also support an electronic circuit, memory, communication circuits and the like to assist with data analysis.

In some embodiments, light source 126 may be configured and arranged to illuminate FRET system 122 with light of a wavelength sufficient to excite a first and/or a second fluorophore of the pair of fluorophores. For example, light source 126 may include at least one of the following: a laser diode, a light-emitting diode (LED), a light bulb (for example, an incandescent light bulb), or bioluminescence (for example, luciferase).

In some embodiments, the sensor embedded in head 104 may include a light source and a wireless integrated circuit. In these instances, the wireless integrated circuit may detect and/or measure fluorescence emission signal 130 when FRET system 122 is exposed to the light source 126, and then transmit fluorescence emission signal 130 to receiving device 128 in response to presence of florescence emission signal 130.

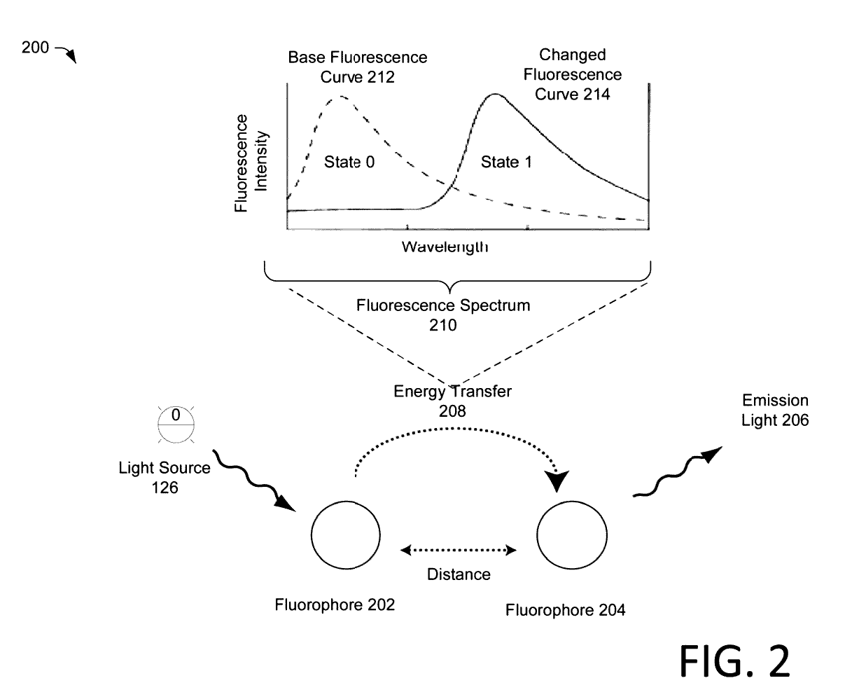

FIG. 2 shows an example scheme 200 illustrating mechanism of FRET system 122, arranged in accordance with at least some embodiments described herein. FRET system 122 may include a pair of fluorophores: a first fluorophore 202 and a second fluorophore 204. According to FRET, when fluorescence donor D (for example, fluorophore 202) and fluorescence acceptor A (for example, fluorophore 204) are close enough, the excited fluorescence donor D will transfer some energy to the fluorescence acceptor A, causing the fluorescence acceptor A to be excited and emit light at a longer wavelength. Energy transfer 208 may be determined based on a fluorescence spectrum 210.

For example, fluorophore 202 may include fluorescein isothiocyanate (FITC), and fluorophore 204 may include tetramethyl rhodamine isothiocyanate (TRITC)-dextran. FRET system 122 may also include concanavalin A (Con A), which is a protein capable of specifically binding to a glucose structure. As for glucose, the binding ability of a single glucose molecule to Con A is stronger than that of fluorophore 204 to Con A. Therefore, in State 0, Con A and fluorophore 204 are in a binding state; so fluorophore 202 cannot transfer energy to fluorophore 204, representing a base fluorescence curve 212 if excited by light source 126.

In state 1, when glucose molecules are present, the binding ability of glucose molecules to Con A is stronger than that of fluorophore 204 to Con A, and the glucose molecules will replace fluorophore 204 to bind to Con A to make fluorophore 204 released. Fluorophore 202 may transfer energy to fluorophore 204 when excited. Thus, the wavelength and intensity of the fluorescence curve change, as illustrated in a changed fluorescence curve 214. A concentration and/or an amount of glucose in the tears may be calculated by a computing device including one or more processors according to changes in fluorescence (For example, time, intensity and wavelength) between state 0 and state 1.

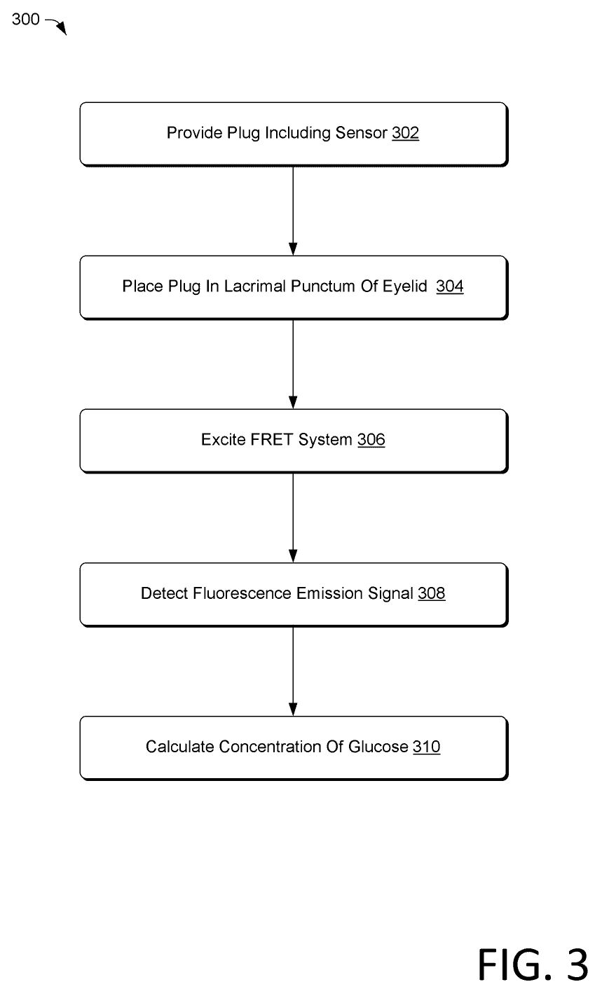

FIG. 3 shows an example processing flow 300 with which a concentration of an analyte in tears of a subject may be calculated and monitored, in accordance with at least some embodiments described herein. Processing flow 300 may be implemented by a user, for example, subject 116, using plug 102 and/or FRET system 122 as described above. Further, processing flow 300 may include one or more operations, actions, or functions depicted by one or more blocks 302, 304, 306, 308 and 310. Although illustrated as discrete blocks, various blocks may be divided into additional blocks, combined into fewer blocks, or eliminated, depending on the desired implementation. Processing flow 300 may begin at block 302.

At 302, processing flow 300 may involve providing a plug with sensor. For example, plug 102 including a sensor described above with respect to FIG. 1 may be provided to subject 116.

At 304, processing flow 300 may involve placing the plug in lacrimal punctum of an eyelid. For example, subject 116 may place plug 102 in the lacrimal punctum of eyelid 114.

At 306, processing flow 300 may involve exciting a FRET system. For example, subject 116 may excite FRET system 122.

At 308, processing flow 300 may involve detecting fluorescence emission signal. For example, receiving device 128 may detect a fluorescence emission signal 130.

At 310, processing flow 300 may involve calculating concentration of glucose. For example, the concentration of glucose in tears in eyelid 114 may be calculated by receiving device 128 based on the fluorescence emission signal 130.

As an illustration, under the scheme of processing flow 300, subject 116 may be provided plug 102, which may include a sensor adapted for measurement of a concentration of an analyte in tears. In some embodiments, the sensor may include FRET system 122 indicative of presence of the analyte in the tears. Plug 102 may be placed in a lacrimal punctum of eyelid 114. In these instances, the analyte may include glucose, and the subject is a human and has diabetes.

FRET system 122 may be excited using light source 126, and fluorescence emission signal 130 may be detected and/or measured. Then, a concentration of the analyte in the tears may be calculated based on the fluorescence emission signal 130.

In some embodiments, FRET system 122 may include a pair of fluorophores such that FRET system 122 generates fluorescence emission signal 130 when fluorophore 202 of the pair of fluorophores is excited by light source 126 and an analyte binding moiety of FRET system 122 binds the analyte. In these instances, fluorophore 204 of the pair of fluorophores non-covalently binds with the analyte binding moiety. In some embodiments, the pair of fluorophore may include at least one of the following: rhodamine and fluorescein isothiocyanate (FITC), tetramethyl rhodamine isothiocyanate (TRITC) and FITC, or tetramethylrhodamine (TAMRA) and FITC-dextran.

In some embodiments, FRET system 122 may be encapsulated into a membrane including physiologically compatible porous nanostructures such that FRET system 122 may be substantially retained on or within the physiologically compatible porous nanostructures. In some instances, the physiologically compatible porous nanostructures may include FMSN.

In some examples, a device for monitoring tears comprises a plug, such as a punctal plug adapted for placement in a lacrimal punctum of a subject, and a sensor. The sensor, such as an optical sensor, such as a fluorescence sensor, may be associated with the plug, and for example may be associated with a head of the plug. For example, the sensor may be configured to be contact with the tears when the plug is installed in the in a lacrimal punctum of the subject. The sensor may be configured for measurement of a concentration of an analyte in the tears. A sensor may comprise one or more materials that provide an optical response that is correlated with a presence of an analyte. In some examples, a plug may comprise a rod-like stem portion (in some examples with a diameter of 0.5 mm-0.8 mm) and in some examples may be configured so that at least a portion of the stem portion may be implanted into a lacrimal punctum of a subject. In some examples, a plug may at least partially, or substantially obstruct the entrance of a lacrimal duct, so as to slow or substantially prevent the flow of tears away from the eyes. In some examples, a plug may be configured to allow some flow of tears along a lachrimal duct. In some examples, a plug may comprise a polymer, such as a silicone polymer (such as polydimethylsiloxane or a derivative thereof or other polysiloxane polymer, other silicon-oxygen backbone polymer, silicone rubber, and the like), a biopolymer (such as collagen), or a thermopolymer (such as polypropylene). The polymer may be or comprise an elastomer. In some examples, a plug may comprise an aerogel (such a silica aerogel), nanoparticles (such as fluorescent mesoporous silica nanoparticles (FMSN)), or other materials. In some examples, implantation of the plug is reversible.

In some examples, a plug comprises a head portion and a stem portion (or anchor portion). The stem portion may comprise a spindle or rod-like portion, and may be configured so at least a portion of the stem portion may be implanted into a lacrimal punctum. In some examples, a stem portion may further support lateral projections, for example from the rod-like portion, and extending from the stem portion to help secure of the plug when the plug is implanted. In some examples, the stem portion may be threaded or comprise grooves or other structures that facilitate compression. The stem portion and/or projections therefrom may be elastically compressed during implantation, and once implanted the stem portion may expand laterally to at least partially block the flow of tears. A head portion may be located proximate the opening of the lacrimal punctum when the plug is implanted, and may be configured to be in contact with tears. The head portion may include, support, or otherwise be associated with a sensor configured to detect one or more analytes in the tears, such as a sensor configured to detect tear glucose. In some examples, a sensor may be located in a stem portion, for example a portion of the stem portion exposed to tears when the plug is impanted, or between the head and stem. In some examples, a sensor may include one or more optical materials (such a fluorophors) disposed on a surface of the head portion, or dispersed within a material forming the head portion. In some examples, a plug may be at least partially supported under a lower eyelid.

In some examples, a plug such as described herein may be implanted into a lacrimal punctum. An external light source may be used to illuminate the sensor associated with the plug and detect an optical signal generated by the sensor in response to the illumination. One or more parameters (such as wavelength, intensity, polarization, decay time, and the like) of an optical signal may be correlated with the presence and/or concentration of the analyte. In some examples, the optical signal is a fluorescence signal. In some examples, a color change associated with the presence of the analyte may be detected. The optical signal may include a visible signal. In some examples, the optical signal may include IR components. In some examples, the sensor may be irradiated sequentially with different wavelengths of light (which may be obtained, for example, from a plurality of light sources, such as LEDs, including one or more near-IR, red, orange, yellow, green, blue, violet, and/or UV light sources. One or more photodetectors may be used to detect the optical signal. In some examples, the light sources may be disposed around one or more photodetectors, for example in a hand-held device. In some exa p, the optical properties of the sensor may be characterized in terms of optical signal intensity as a function of irradiating wavelength. In some examples, a fluorescence parameter such as fluorescence intensity, a fluorescence wavelength, and/or a fluorescence lifetime may be detected. Fluorescence lifetimes may be determined by detecting the time-dependence (for example decay) of an optical signal such as fluorescence after pulsed illumination by one or more light sources, or from phase differences between irradiation and emission signals. In some examples, the sensor may be sensitive to a plurality of analytes, and for example fluorescence from a plurality of fluorophors may be detected by sequentially illuminating the sensor with pulses of different wavelengths of light and detecting the corresponding emissions. In some examples, a plurality of fluorophors may be used to detect the same analyte, to improve accuracy.

By “about” it is meant a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length

As used herein, the terms “patient”, “subject” and “individual” are used interchangeably herein, and mean a mammalian subject to be treated and/or to obtain a biological sample from. Mammalian subjects may include humans and domestic animals, such as cats, dogs, swine, cattle, sheep, goats, horses, rabbits, and the like.

“Substantially” means nearly totally or completely, for instance, 95%, 96%, 97%, 98%, 99% or greater of some given quantity.

As used herein, the terms “diagnostic”, “diagnose” and “diagnosed” mean identifying the presence or nature of a pathologic condition.

By “analyte” is meant any molecule or compound (for example, glucose). An analyte can be in the solid, liquid, gaseous or vapor phase. The term analyte may include polynucleotide analytes such as those polynucleotides defined below. These include m-RNA, r-RNA, t-RNA, DNA, DNA-RNA duplexes, etc. The term analyte also includes receptors that are polynucleotide binding agents, such as, for example, peptide nucleic acids (ARIA), restriction enzymes, activators, repressors, nucleases, polymerases, histones, repair enzymes, chemotherapeutic agents, and the like. The analyte may be a molecule found directly in a sample such as a body fluid from a host. The sample can be examined directly or may be pretreated to render the analyte more readily detectable. Furthermore, the analyte of interest may be determined by detecting an agent probative of the analyte of interest such as a specific binding pair member complementary to the analyte of interest, whose presence will be detected only when the analyte of interest is present in a sample. Thus, the agent probative of the analyte becomes the analyte that is detected in an assay. The body fluid can be, for example, urine, blood, plasma, serum, saliva, semen, stool, sputum, cerebral spinal fluid, tears, mucus, and the like.

It will be understood by those within the art that, in general, terms used herein, and especially in the appended claims (for example, bodies of the appended claims) are generally intended as “open” terms (for example, the term “including” should be interpreted as “including but not limited to,” the term “having” should be interpreted as “having at least,” the term “includes” should be interpreted as “includes but is not limited to,” etc.” It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an,” (for example, “a” and/or “an” should be interpreted to mean “at least one” or “one or more”) the same holds true for the use of definite articles used to introduce claim recitations. In addition, even if a specific number of an introduced claim recitation is explicitly recited, those skilled in the art will recognize that such recitation should be interpreted to mean at least the recited number, (for example, the bare recitation of “two recitations”) without other modifiers, means at least two recitations, or two or more recitations. Furthermore, in those instances where a convention analogous to “at least one of A, B, and C, etc.” is used, in general such a construction is intended in the sense one having skill in the art would understand the convention, for example, “a system having at least one of A, B, and C” would include but not be limited to systems that have A alone, B alone, C alone, A and B together, A and C together, B and C together, and/or A, B, and C together, etc. In those instances where a convention analogous to “at least one of A, B, or C, etc.” is used, in general such a construction is intended in the sense one having skill in the art would understand the convention, for example, “a system having at least one of A, B, or C” would include but not be limited to systems that have A alone, B alone, C alone, A and B together, A and C together, B and C together, and/or A, B, and C together, etc. It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

Lastly, with respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the Following claims.

1. A device for monitoring tears, the device comprising:
a plug adapted for placement in a lacrimal punctum of an eye of a subject; and
a sensor that is associated with a head of the plug and is in contact with the tears, the sensor comprising a fluorescence resonance energy transfer (FRET) system to detect a presence of an analyte in the tears.

2. The device of claim 1, wherein the analyte comprises glucose, and wherein the subject is a human having diabetes.

3. The device of claim 2, wherein the FRET system comprises a pair of fluorophores such that the FRET system generates a fluorescence emission signal when:
a fluorophore of the pair of fluorophore is excited by a light source; and
an analyte binding moiety binds the analyte, the analyte binding moiety being integrated in the FRET system and associated with an additional fluorophore of the pair of fluorophores.

4. The device of claim 3, wherein fluorophores of the pair of fluorophores have at least about 30 nm difference in florescent wavelengths.

5. The device of claim 3, wherein the pair of fluorophores comprises at least one of rhodamine and fluorescein isothiocyanate (FITC), tetramethyl rhodamine isothiocyanate (TRITC) and FITC, or tetramethylrhodamine (TAMRA) and FITC-dextran.

6. The device of claim 3, wherein the light source is a light-emitting diode (LED).

7. The device of claim 3, wherein the fluorescence emission signal comprises information associated with a fluorescence intensity, a fluorescence wavelength, a fluorescence lifetime, or a combination thereof.

8. The device of claim 2, wherein the FRET system is adapted for a fluorescence-based chromatographic assay.

9. The device of claim 2, wherein the FRET system is encapsulated into a membrane comprising physiologically compatible porous nanostructures such that the FRET system is substantially retained on or within the physiologically compatible porous nanostructures.

10. The device of claim 9, wherein the physiologically compatible porous nanostructures comprise porous medium adapted for collection of the tears.

11. The device of claim 9, wherein the physiologically compatible porous nanostructures comprise fluorescent mesoporous silica nanoparticles (FMSN).

12. The device of claim 2, wherein the sensor comprises a light source and a wireless integrated circuit, and wherein the sensor is configured to perform operations comprising:
detecting a fluorescence emission signal generated by the FRET system when the FRET system is exposed to the light source; and
transmitting the fluorescence emission signal in response to presence of the florescence emission signal.

13. The device of claim 1, wherein the plug comprises at least one of silicone acrylates, silicone derivatives, fluorophore, polymethylmethacrylate (PMMA), polydimethylsiloxane (PDMS), or polydimethylsiloxane.

14. The device of claim 1, wherein the device is a punctal plug adapted for placement in a superior lacrimal punctum or an inferior lacrimal punctum of the eyelid.

15. A method for monitoring tears, the method comprising:
providing a plug comprising a sensor adapted for measurement of a concentration of an analyte in the tears, the sensor comprising a fluorescence resonance energy transfer (FRET) system indicative of presence of the analyte in the tears; and
placing the plug in a lacrimal punctum of an eyelid of a subject.

16. The method of claim 15, wherein the analyte comprises glucose, and wherein the subject is a human having diabetes.

17. The method of claim 15, further comprising:
exciting the FRET system using a light source;
detecting a fluorescence emission signal associated with the FRET system; and
calculating a concentration of the analyte in the tears based on the fluorescence emission signal.

18. The method of claim 15, wherein the FRET system comprises a pair of fluorophores such that the FRET system generates a fluorescence emission signal when a fluorophore of the pair of fluorophores is excited by a light source and an analyte binding moiety of the FRET system binds the analyte, and wherein an additional fluorophore of the pair of fluorophores non-covalently binds with the analyte binding moiety.

19. The method of claim 18, wherein the pair of fluorophores comprises at least one of rhodamine and fluorescein isothiocyanate (FITC), tetramethyl rhodamine isothiocyanate (TRITC) and FITC, or tetramethylrhodamine (TAMRA) and FITC-dextran.

20. The method of claim 15, wherein the FRET system is encapsulated into a membrane comprising physiologically compatible porous nanostructures such that the FRET system is substantially retained on or within the physiologically compatible porous nanostructures.

21. The method of claim 20, wherein the physiologically compatible porous nanostructures comprise fluorescent mesoporous silica nanoparticles (FMSN).

22. A system, comprising:
a plug adapted for placement in a lacrimal punctum of an eyelid of a subject having diabetes, the plug comprising:
a sensor that is associated with a head of the plug and is contact with tears in the eyelid of the subject, the sensor adapted for measurement of a concentration of glucose in the tears, the sensor comprising a fluorescence resonance energy transfer (FRET) system which comprises a pair of fluorophores;
a light source adapted to excite the FRET system to generate a fluorescence emission signal when:
a fluorophore of the pair of fluorophores is excited by the light source, and
an analyte binding moiety associated with an additional fluorophore of the pair of fluorophores binds the analyte; and
a receiver adapted for receiving the fluorescence emission signal.

23. The system of claim 22, wherein the pair of fluorophores comprises at least one of rhodamine and fluorescein isothiocyanate (FITC), tetramethyl rhodamine isothiocyanate (TRITC) and FITC, or tetramethylrhodamine (TAMRA) and FITC-dextran.

24. The system of claim 22, wherein the FRET system is encapsulated into a membrane comprising physiologically compatible porous nanostructures such that the FRET system is substantially retained on or within the physiologically compatible porous nanostructures.

25. The system of claim 24, wherein the physiologically compatible porous nanostructures comprise a porous medium adapted for collection of the tears.

26. The system of claim 24, wherein the physiologically compatible porous nanostructures comprise fluorescent mesoporous silica nanoparticles (FMSN).
